(19) United States
(12) Patent Application Publication
(10) Pub. No.: US 2007/0265571 A1
(43) Pub. Date: Nov. 15, 2007

(54) Needle alignment, needle securement and vessel stabilization device

(76) Inventors: David Utterberg, Seattle, WA (US); David Bell, Grayslake, IL (US); William J. Schnell, Libertyville, IL (US)

Correspondence Address: SEYFARTH SHAW LLP, 131 S. DEARBORN ST., SUITE2400, CHICAGO, IL, 60603-5803, US

(21) Appl. No.: 11/431,137

(22) Filed: May 9, 2006

(51) Int. Cl. A61M 5/32

(52) U.S. Cl. 604/174

(57) ABSTRACT

A securement device for an intravenous, winged needle set, which comprises: an inverted U-shaped, self-supporting strap, the strap having vertical legs attached to a central portion of a base. The base has a bottom to rest on the skin of the patient. A portion of the base forward of the strap has an upper surface that slopes downwardly to a forward end at an angle to the skin and the bottom of the base. Improved retention and ease of application is provided with such a device.

### BACKGROUND OF THE INVENTION

Hollow bore cannulae of a needle set for access to the vascular system of a patient have sharp tips at their distal end which are cannulated through the skin and vessel wall, and such tips reside within the vessel lumen. Often, as is well known, winged hub needle sets are used, one advantage of the wings being that they aid in the practitioner's secure and rigid holding of the device during cannulation, and then because of their flexibility may be flattened onto the skin and provide a taping aid to tape the device in place on the skin.

Particularly in the case of winged needle sets for hemodialysis and other extracorporeal procedures, a pair of winged needles must be placed in a very secure position on the surface of the skin, since the sharp cannula tips reside in the fistula or graft for a substantial period of time, and a great deal of blood could be lost if either needle de-cannulates from the patient, since a dialysis machine is pumping blood through the needles. Additionally, significant trauma to the cannulated vessel occurs if the cannula is not maintained at an angle relative to the plane of the skin which prevents the sharp needle tip from touching or piercing the lateral or posterior wall of the cannulated fistula or graft. Fistulae, grafts and arteries are not the superficial veins of many simple intravascular procedures, but may lie beneath 3-15 mm of tissue. Depending on the depth and internal diameter of the vessel the dwell angle for the cannulated needle must typically about be 1-30 degrees to the plane of the skin to reach the vessel, but not so angled that the sharp needle tip is in danger of touching or piercing the lateral or posterior vessel wall.

Another aspect of surgically inserted fistulae and grafts is that they have cannulating portions along their length that may be transverse to the axis of the limb they inhabit, or at other angles to the axis of said limb, based on the surgeon's placement of such fistula or graft. Cannulation of such portion must be along the axis of such portion which is not necessarily parallel with the axis of said limb (versus most intravenous cannulations of, for example, the occipital vein, which is essentially parallel to the axis of the limb). Additionally, the skin overlaying such cannulatable portions of the vessel can be very irregular, resulting from the surgical procedure and growth of tissue surrounding such fistula or graft. Thus, the securement of the needle/hub to the skin must be at a variety of angles relative to both the plane of the skin or the axis of the limb at the cannulation site, and securement must be made sometimes to very irregular skin surfaces.

Another issue of arterial or fistulae/graft cannulation is that blood can spontaneously leak from around the cannulated needle at the cannulation site at any time during the 3-4 hour dialysis or other extracorporeal procedure, in which typically the patient is anticoagulated.

In the prior art, significant amounts of tape are typically attached between the tubing, wings, and hub of winged needles, and the skin, in efforts to immobilize the sharp needle tip. Gauze or other materials are often placed under the hub or tubing, to maintain the cannulated needle at a proper dwelling angle for the non-superficial vessel. In the most current prior art, at least one of these pieces of tape, typically, is provided by a known, “chevron-style” taping method, typically viewed as the best available method. This method first attaches the sticky side of a 4-5″ strip of tape at its middle to the underside of the tubing just proximal to the winged hub, and thence crossing each extension leg of the tape back over the top of the tube in criss-cross manner, and then over the top of each wing, and then onto the skin distal to the wings, for securance of the device to the skin.

However, difficulties can arise with this and other taping styles for long dwell winged hub needle sets. One difficulty is the inherent flexibility of all the principal components of the taping method: the wings and tubing themselves, the skin, and the tape. The hub and cannula are rigid, but are anchored by flexible components that at best can allow the sharp cannula tip to move about within the vessel and at worst to loosen over time to expand the range of cannula swing by continuous movement of the patient or tubing.

Another difficulty results from movements during the tape application itself. As the tape is applied to the tubing and/or winged hub, the external (uncannulated) portion of the rigid hub and cannula may be urged in any direction. Such external movement is translated via a leverage point at the cannulation site into opposite movement of the sharp cannula tip within the vessel, often resulting in internal laceration of the vessel or even infiltration of the lateral or posterior vessel wall.

Another difficulty occurs when, typically, the back end of the winged hub is propped up off the skin surface by some millimeters, typically by the clinician's multi-folding of a piece of gauze to the desired thickness, in attempt to hold the external cannula/hub at a 15-30 degree angle for deeper vessels, with the clinician's goal of keeping the sharp cannula centrally located in the vessel during the entire procedure (i.e. away from the vessel walls). However, the placement of this gauze often inadvertently urges the external cannula/hub upward, with the attendant movement of the internal, sharp needle tip downward, also risking infiltration and laceration. Also, such hand-folded gauze is of a different thickness each time, and the folds themselves are a kind of spring that are urging gauze reopening. Such reopening also urges the external cannula/hub upwardly, with internal sharp tip movement downward.

Another difficulty of the prior art in hemodialysis and other two-needle procedures is the tube that connects to the winged needle hub often must be manipulated by the clinician into a U-shape or S-shape, the curvature of which is often severe, and must start curving directly adjacent the tubing/hub connection in order to avoid the cannulation site of the other of the cannulated winged needle sets or other medical instrumentation. Such manipulation and tight curves result in potential energy stored in the curved tubing, which urges or seeks to urge movement of the needle tip in the vessel.

The prior art includes numerous devices for holding external segments of intravenous needles, but each of these provide incomplete solutions, or cause their own problems. For example, Hakky U.S. Pat. Nos. 6,113,577 and 6,500,154 disclose a securement device for winged needle sets which comprises a shield covering a length of the needle, hub, and tubing, and is secured to the patient by a strap wrapping around the cannulated limb. Covering the tubing, even partially with a rigid plastic as disclosed, prevents curving of the tube as required in some cannulation placements, and in any case essentially increases a lever length, adding to the force urging movement of the needle tip when the tubing is manipulated into curved orientation. The limb-surrounding strap limits the device to only those cannulation sites wherein the underlying vessel has a cannulating portion that is parallel to the axis of the limb. Additionally, though Hakky discloses the possibility of use in dialysis, the disclosed device has no provision for maintaining any dwell angle of the cannulated needle other than zero angle. Further, by the use of a rigid plastic, there is no ability for the nurse to modify shape of the shield to conform better to the shape of the arm or irregular skin surfaces. Finally, the pressure exerted by a limb-encircling strap may be such to actually occlude the underlying vessel. As such occlusion is associated with stenotic injury, DOQI, ANNA and other learned dialysis organizations warn against use of tourniquets, clamps or any other limb-encircling devices on a limb containing a fistula or graft.

The prior art also includes many devices that include adhesive ingredients for direct adherence of such device to the skin. However, since all adhesives yet identified are such that at least some patients are allergic to, it is a problem rather than a solution for medical devices to be pre-equipped with adhesive, thus preventing the device's use on patients allergic to the particular adhesive. One such example is of a device for wingless catheters by Bierman U.S. Pat. No. 7,014,627.

A separate but related aspect of winged needle sets for cannulation of deep vessels, fistulae, grafts and the like relates to the difficulty for the clinician to accurately penetrate the vessel lying 3-15 mm underneath the opaque skin at a particular point along the topmost surface of such vessel's cannulating portion, said particular point being typically 10-35 mm away from the cannulation site at the surface of the skin, said 10-35 mm being the length of the needle track between the skin surface and the cannulation point on the vessel wall. Also, because of the typically 1-30 degree initial cannulation angle and such a “blind” needle track between the cannulation target on the skin surface, such cannulation requires skilled practitioners. Especially in the case of fistulae or grafts, cannulation is made difficult due to the tendency of a fistula, especially a maturing fistula or recently implanted graft, to move laterally under the skin away from the needle tip advancing down a needle track through the overlying tissue. This often results in mis-cannulations or mal-cannulations of the fistula or graft, resulting in injury to the vessel. To secure the fistula from such lateral movement, the clinician often grasps, with thumb and forefinger of the non-dominant hand, the skin overlaying the fistula right at the intended particular vessel point for the needle stick (which is separated from the external cannulation site by only the length of the subsequent needle track) with the goal of immobilizing the particular vessel point from such lateral movement. However, this is a dangerous maneuver. First, since the clinician's fingers are at risk of accidental needlesticks from the sharp, advancing needle tip, and second, fingers are round in cross section, thus preventing intimate contact with the underlying vessel that is also round, but curving in the opposite directions from each finger. This results in less than perfect stabilization of the fistula.

By this invention, a device and methods for securing a winged needle set after cannulation and desired cannula angle alignment are provided, plus methods for immobilizing from lateral movement the vessel prior to and during cannulation by said winged needle set. By this invention, a simpler form of taping may be used, which is nevertheless highly reliable and avoids urging of the sharp cannula tip in a lacerating direction, allowing use of any tape adhesive with which the particular patient is compatible.

By this invention, said device can hold the cannula at a fixed angle to the skin conducive to maintaining the cannula tip within the vessel away from the vessel walls, and said device can also provide added needle retention and stabilization, despite movement of the tubing into U-shaped or S-shaped manipulations in any direction. By this invention, said device may be rigid but malleable, and is placed between the wings and patient's skin, preferably after cannulation, providing a rigidifying structure between the flexible wings, tubing, and the flexible skin that increases the security of taping, also avoiding risk of cannula pull-out.

The fistula needle securement and alignment device of this invention permits taping in which the tape is applied in straight strips over the wings, in a manner generally longitudinal relative to the needle axis, holding the wings down on the rigidifying device, without need of complicated, chevron taping. This is, of course, much simpler to apply than chevron taping, requiring less skill, and avoiding movement of the tubing or winged needle hub. Nevertheless, through the use of the needle securement device, significantly increased pull force (i.e. the amount of pull required to remove the needle from the patient) can be obtained, with adequate room for shaping the tubing connected to the needle as it is taped to the skin in, for example, a U-shape or an S-shaped configuration depending upon the situation.

The winged needle set may be secured without significant covering of the cannulation site or external segments of the cannula, allowing for placement of gauze or other devices to control leakage around the cannula at such cannulation site.

Typically, the device may be resilient enough to provide securement of the needle device, but is bendable by the clinician so that its shape may be adapted, without much elastic memory, to be shaped to change the angle at which the winged needle set may dwell relative to the skin surface at its chosen site as well as to deform the device shape in other ways to adapt to the patient's particular cannulation site.

Securement of the winged needle set at the required cannulation angle may be at any position and at any angle to the axis of the cannulated limb.

Finally, by this invention, the same or similar device can be used by the clinician during cannulation in stabilization of the fistula under the skin against lateral movement, to allow the clinician's stabilization fingers to grasp the device some distance away from the cannulation site rather than the skin directly at said site, thus providing a significant degree of protection against accidental needle sticks.

### DESCRIPTION OF THE INVENTION

By this invention, a device is provided for securing a winged needle set that penetrates the skin of a patient against motion of the cannula of said set within the skin, to protect particularly the blood vessel in which the cannula resides. The device comprises: a base for placement on the skin of the patient, and for immovably holding the winged needle set while the cannula penetrates the skin, and the wings of the needle set are substantially separated from the skin, typically by the device. In some embodiments, the wings of the needle set may be carried on the base in contact therewith, in a manner spaced from the skin.

Generally, a “needle” or “needle set” comprises a cannula and a hub. A “cannula” refers to the skin-piercing metal tube, typically without reference to the hub.

Further by this invention, an alignment and securement device for an intravenous, winged needle set is provided, which comprises: an inverted typically U-shaped, self-supporting strap, the strap having substantially vertical legs attached to a center section of a base. The strap defines an opening through which the tube of the intravenous needle device or even sections of its hub and/or wings may pass through. Preferably, the opening is essentially no more than the outer dimension of the section of tube and/or hub that lies within such opening. The strap legs preferably are no wider than about 5 mm (in the needle axis direction) so as to leave as much as possible of the needle set tubing to be unencumbered by said strap after device placement so the secured tubing may be curved according to clinical needs. The base defines a bottom, at least portions of which rest on the skin of a patient. At least a portion of the base forward of the strap has a first, upper surface that slopes from the strap downwardly to a forward end, at an angle of essentially 1-30°, typically at least about 5°. The portion preferably is not substantially less in size than the wings of a winged needle set with which the device is to be used. Thus, when a cannulated, winged needle is laid upon that first, upper surface, it naturally assumes the same angle to the skin as is defined by the first, upper surface, so that the needle placed thereon and cannula inserted into the skin tends to assume that desired angle. In some embodiments, the angle of the above first, upper surface is essentially 5-10°, or 10-15° to the skin surface on which it is laid.

Both of these stated angle ranges are relative to the skin under the securement device. That is, when the securement device is positioned on flat skin, the first, upper surface will be at an angle to the skin. That is the angle which may be in one or more of the above angle ranges, and is essentially the angle that a cannula of a winged needle set carried thereon should enter the skin.

The device may be made of opaque plastic, so that it is readily distinguishable from the skin and winged needle set from a good distance by the clinician. Alternatively, it may be transparent.

In some embodiments, the base of the securement device may be a single, unitary base, or the base may divided into a pair of separate, spaced segments, separated by a complete space so that the base is in two, spaced segments. The space extends under the U-shaped strap, and may be of a width to at least partly receive a tube and/or hub of the winged, intravenous needle. Thus, the winged needle set may be cannulated as normal and placed initially at its desired dwell angle without reference to said device. Then, the device may be placed down over the tubing some millimeters behind the hub of the needle set, with at least part of the tubing positioned resting within said strap opening. Then the device is slid forward so the two, spaced segments slide under the pair of wings, so the wings of said hub are resting on the upper surfaces of the base without bending or distortion. Preferably, the forward edge of the strap just touches the back edge of each wing. The wings may be then taped to this device, as well as the wings and device to the skin, while the wings are resting on the first, upper surface. The tape may be applied, with great ease and with the need of less skill, in a longitudinal manner relative to the needle axis along the securement device and the wings, with the tape adhering to the wings, the skin, and preferably the device, without the need of an elaborate and fairly difficult taping technique such as the chevron style, with its attendant urgings of the needle tip to cause lacerations or infiltrations. In this position not only is the hub and rigid cannula held at an essentially precise angle to the skin, but the wings may not move backward despite a pulling force on the tubing or wings because of the preferable engagement of the strap's forward edge to the back edge of the wings, such engagement being preferably directly adjacent the hub where the connected wing is essentially inflexible. Further, the inverted U-shaped strap prevents clinician's lifting or bending of the tubing from transmitting leveraging force to the sharp cannula tip. Thus, the practitioner may manipulate the tubing after needle securement according to the clinical needs, and the patient is free to personally move as often as necessary during the long treatment, without fear of causing a laceration or infiltration of their precious fistula lifeline.

A portion of the base behind the strap may have a second, upper surface(s) that slopes downwardly to a rearward end. This provides a tape-receiving surface for the securance tape that also extends over the wings of a needle carried on the securement device, so that single strips of longitudinally extending tape adhere to the securement device, to the wings of the needle carried thereon, and to the skin. Typically, a pair of such longitudinal tape strips are provided, one on each side of the emplaced, winged needle set and each side of the U-shaped strap.

In some embodiments, the second, upper surface may be of a different angle to the skin than the first, upper surface, and may be used with the device reversed in orientation such that the second, upper surface of the base is forward of the strap, and is used as the wing resting surface. For example, for some deep fistulas, an angle of about 15-20 degrees for the indwelling needle may be more ideal than a lesser angle (of the first, upper surface) and so such second upper surface(s) with about a 15-20 degree angle may be used to provide resting and securement surfaces for the wings, while the first, upper surface(s) directly adheres to strips of retaining tape for retention, like that described above.

In some embodiments, the second, sloping, upper surface has a portion of maximum height that is adjacent to the strap, and is higher than the bottom of the strap legs where they join to the base, typically higher by an amount that is about the thickness of the wings to be placed on the first upper surface(s). Thus, tape that is applied can smoothly extend over the second, upper surface, and then over the wing, without a disjunction or discontinuity at that point. The same tape also may extend over the skin at both tape ends, and being adhered thereto both forward of and to the rear of the securement device and the carried needle wing; with the winged needle hub positioned under the U-shaped strap, and with wings of the needle resting on the first, upper surface, or alternately the second, upper surface, and secured there by the tape.

Furthermore, in some embodiments, the device may be advanced until a forward edge of each leg of the U-shaped strap may abut the back of the wings, close to said wings' joining points to the hub where the wings may be least flexible. This provides further securance of the winged needle and the securement device, above and beyond the securance that the taping provides.

Furthermore, in some embodiments, the material of construction of the device has a bendability that allows some modification of the constructed shape to be made by the clinician to change the angle between the forward and rear segments, to adjust the horizontal angle of such segments on a limb (where the skin is curved such as on a small arm), to adjust the base to an angle that conforms to the patient's skin or to conform to the desired cannula angle.

The term “inverted U-shaped strap” may also include straps of inverted V shape, rectangular arch shape, and the like. The strap cross section may be rectangular, round, oval, or the like, generally without limitation.

Accordingly, the securement device may be secured to the skin of a patient, with the winged needle carried thereon, with the securance being typically provided by separate lengths of securance tape, each extending over a wing of the winged needle, while the tape preferably extends in a direction generally longitudinal to the cannula axis, with the length of tape having end sections adhering to the skin while leaving the attached, flexible tubing capable of extensive movement, while such movement does not translate into movement of the sharp cannula tip within the cannulated vessel.

This invention may also be used as a fistula stabilization device to make cannulation safer and less prone to mal- or mis-cannulation due to lateral movement of the fistula underlying the skin. In a typical embodiment, the device may be the same design as the securement device but used in a different way for a different purpose. The device is held typically by the U-shaped strap in the clinician's non-dominant hand, such that the space between the base segments straddles the patient's vessel to be cannulated, typically a fistula. Preferably, the cannulation target is near the end of the base segments. With light downward pressure from the clinician, the fistula can be relatively immobilized from lateral movement by the device, thus making cannulation by the clinician's dominant hand less prone to mal- or mis-cannulation, and less prone to the clinician receiving an accidental needle stick to the non-dominant hand, that in the prior art typically stabilizes the fistula by directly touching the patient's skin within millimeters of the cannulation target.

Also, in some embodiments, the base may define a transverse and/or axial line of bending weakness (i.e. generally transverse to the axis of a needle placed on the device) to permit manual bending of the base to form two typically obtusely angled sections out of a generally flat bottom. By this technique, the angle of the first, upper surface, as well as the second upper surface, to the skin of the patient on which the device rests may be adjusted, to account for the corresponding angle to the skin of a needle carried on the securement device. The securement device may be taped to the skin while occupying the desired angle of the two angled sections of the generally flat bottom, being held in that position by the tape on the skin which, as before, may be taped in longitudinal strips over the wings and the securement device.

Typically, the material of which the securement device may be made, generally as a one-piece molding, is a material which is bendable along the line of bending weakness, and not strongly resilient, so that the bend of the material tends to stay in the desired angle of bend which may be imposed on it. Plastics such a polycarbonate, impact modified styrene and styrene co-polymers have such bending, low memory attributes.

Further by this invention, a method is provided of stabilizing a blood vessel to facilitate its penetration by a cannula through the skin. The method comprises: applying to the skin a stabilization device which comprises a base having laterally spaced segments, for example as described above, with the blood vessel positioned between the base segments, to limit lateral movement of the blood vessel as the cannula is advanced to penetrate the blood vessel through the skin. The spaced segments may be connected by the inverted U-shaped strap described above, or by any other technique for connection into a generally rigid system. Also, the spaced segments may be parts of a single, unitary base, for example having a bottom-opening, central tunnel portion to receive the blood vessel.

After cannula penetration, the stabilization device may be thereafter repositioned to support and stabilize the needle set, as described above, as said set resides on the skin in skin-penetrating relation, further including the step of taping a hub and/or wings of the needle, the stabilization device, and the skin together to form a relatively rigid composite that greatly reduces movement capability of the cannula penetrating the blood vessel. As before, the blood vessel may comprise a vein, an artery, a fistula, a graft, an implanted blood catheter, or the like.

In some embodiments, the hub carries wings in conventional manner, and the wings of the hub rest, after the taping, on the base portions, with the wings being spaced from the skin. However, wingless needle sets may also be used.

Further in accordance with this invention, a method is provided of stabilizing a cannula residing in a blood vessel and extending through the skin of the patient, comprising: placing a securement device comprising a base onto the skin of the patient adjacent to the needle hub, and taping the skin, the base, and the needle hub together into a substantially rigid composite, as described above, for similar desirable purposes. The wings of the hub, as before, may rest on the base, spaced from the skin.

By this invention, a needle set having flexible wings and flexible tubing attached thereto may be attached to flexible skin by use of the securement device of this invention (which also includes its use as an alignment and blood vessel stabilization device as described above), providing rigidity to the entire system by the use of the device of this invention, greatly reducing the risk of internal blood vessel laceration by the tip of the cannula, and providing a desired, stable angle of entry by the cannula to the blood vessel and reduced risk of cannula pull-out from the patient.

### DESCRIPTION OF THE DRAWINGS

In the drawings, FIG. 1 is a perspective view of an embodiment of the alignment and securement device of this invention, carrying an intravenous, winged needle with the cannula penetrating the skin of the patient, the device being taped onto the skin by longitudinally extending lengths of tape, each tape position being schematically shown as a line.

FIG. 2 is a perspective view of the alignment and securement device of FIG. 1, shown by itself without the needle set or tape.

FIG. 3 is a plan view of the alignment and securement device of FIG. 2.

FIG. 4 is a rear elevational view of the alignment and securement device of FIGS. 2-3.

FIG. 5 is a side elevational view of the alignment and securement device of FIGS. 24.

FIG. 6 is a perspective view of the device of FIGS. 1-5, carrying a winged needle and taped on the skin.

FIG. 7 is a perspective view of another embodiment of this invention.

FIG. 8 is a perspective view of a third embodiment of this invention.

FIG. 9 is a plan view of the device of FIG. 8.

FIG. 10 is an elevational view of the device of FIGS. 8 and 9, shown resting on the skin, with the tape and winged needle removed.

FIG. 11 is a perspective view of another embodiment of this invention.

FIG. 12 is a side elevational view of FIG. 11.

### DESCRIPTION OF SPECIFIC EMBODIMENTS

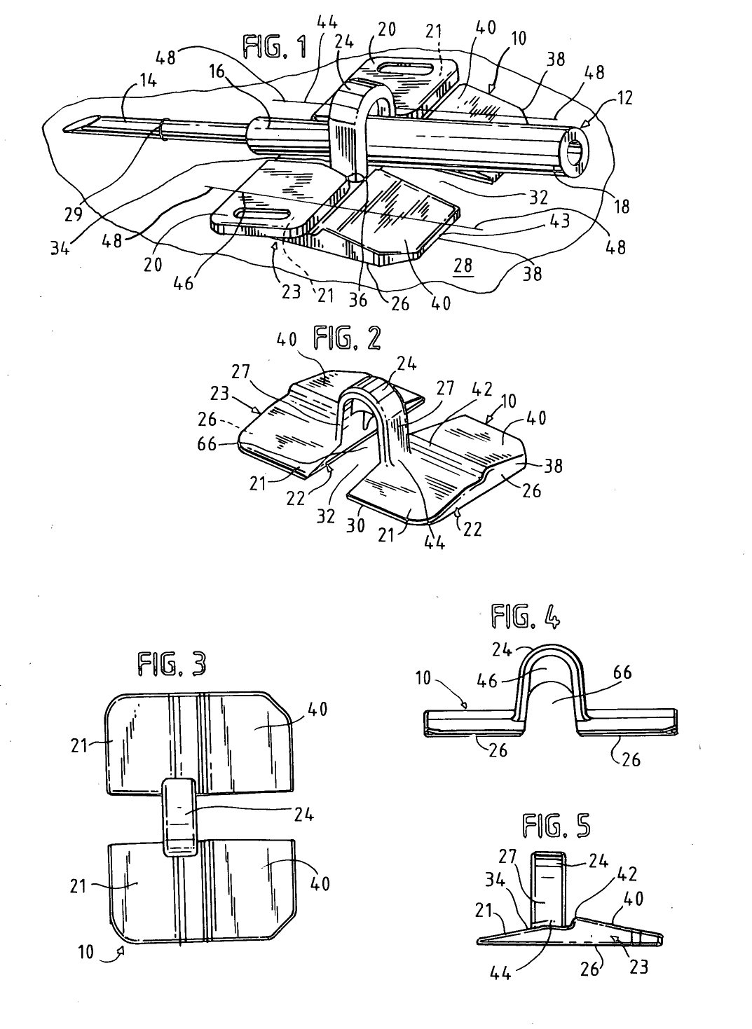

Referring to the drawings, FIGS. 1 and 2 show an alignment and securement device 10 which carries an intravenous, cannula 14 as part of a hemodialysis winged fistula needle set 12. Needle set 12 comprises cannula 14, needle hub 16 and flexible tubing 18, which typically terminates in a conventional tubing connector (not shown). Extending from hub 16 are a pair of flexible wings 20, of conventional design.

Wings 20 are shown to be resting at an alignment angle on a first, angled upper surface 21 of a forward portion 22 of base 23 of alignment and securement device 10. It can be seen that, in this embodiment, the base 23 of alignment and securement device 10 is divided into two segments 22 (FIG. 2), held together by inverted U-shaped, self-supporting strap 24. Thus, it can be seen that alignment and securement device 10 can be molded as a single, integral plastic piece, being typically at least essentially semi-rigid, so as to hold its configuration without much flexing or shape change, unless deliberately bent to a new desired shape.

U-strap 24 defines a pair of substantially vertical legs 27 (FIG. 2), which join to each segment 22 of base 23 and defines an opening 66 (FIG. 2). Base 23, whether unitary or in its two segments as specifically shown, defines a generally flat bottom 26 to rest on the skin 28 of the patient, as shown in FIG. 1, cannula 14 penetrating the skin at site 29. Preferably the material of construction is bendable so the base 23 can be shaped by the clinician for best fit to the skin 28. Typically, the first, angled, upper surface 21 of each forward portion 22 slopes downwardly toward the actual forward end edge 30 (FIG. 1) at a typical angle of essentially 1-20 degrees for the alignment of the cannula 14 into the cannulated vessel; in some embodiments the angle being essentially 5-10 degrees, or 10-15 degrees.

Space 32, between the segments 22 (FIG. 2) of base 23 and contiguous with opening 66 under strap 24, divides the pair of segments 22. Space 32 may be of a width to a least partly receive hub 16 and/or flexible tubing 18 of the winged, intravenous needle 12 carried thereon. That is, at least the bottom of hub 16 and/or flexible tubing 18 can pass through opening 66, and may project into space 32, to improve the flat seating of wings 20 on first, angled, upper surfaces 21. Preferably the fit of hub 16 or tubing 18 in space 32 and opening 66 is snug, to restrain motion of the hub and cannula 14.

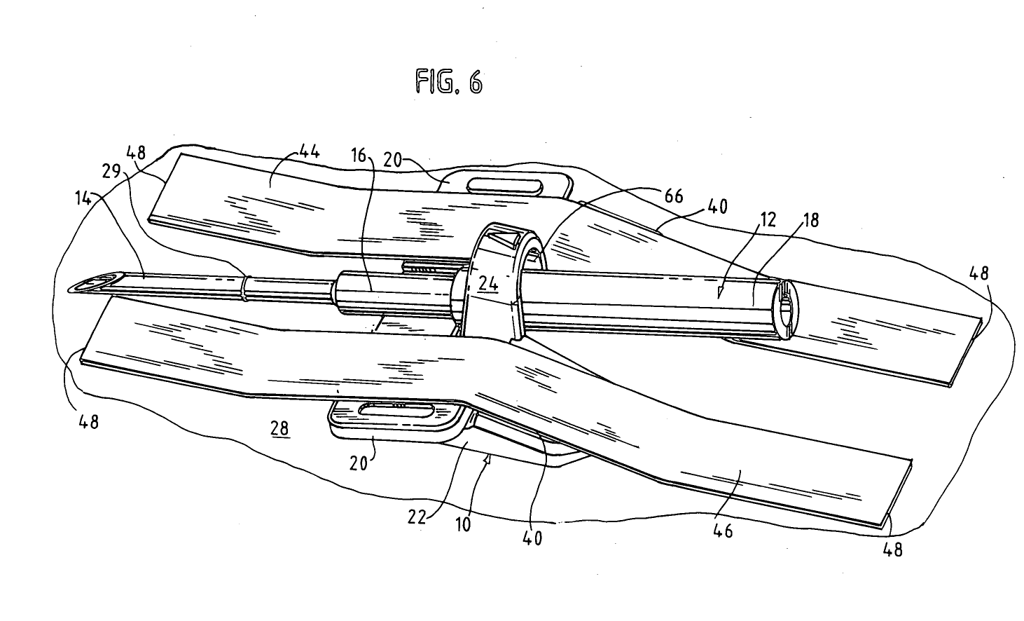

FIG. 1. shows the winged needle set 12 after cannulation into the patient, and after placement of alignment and securement device 10 and engagement with winged needle set 12, but prior to taping of same to the patient's skin 28. Prior to the arrangement shown in FIG. 1 the winged, needle set 10 is cannulated according to the prior art, and with alignment and securement device 10 preferably being either not carried on said set 10 or carried on tubing 18 away from hub 16 so as to not interfere with cannulation of the patient. After cannulation and initial alignment of cannula 14 and hub 16, the alignment and securement device 10 of FIGS. 2-5 is grasped by the clinician, and the space 32 and opening 66 of strap 24 is engaged lightly over tubing 18 with each of forward segments 22 placed well behind each of wings 20. Then the alignment and securement device 10 is slid forward such that the first, angled upper surfaces 21 slide under each of wings 20 until the forward edge of strap 24 preferably lightly abuts the back edges 36 of wings 20. Then the alignment and securement device 10 and winged needle set 12 of FIG. 1 can be taped along lines 44, 46 according to physician's prescription, or as shown in FIG. 6 to patient's skin 28.

In FIG. 1, the pair of bottom segments at forward edge 34 of strap 24 are each abutted against the back edge 36 of wing 20 at or near the joint between each wing 20 and needle hub 16, with the inner dimension between the vertical legs 27 being close to the outer dimension of hub 16 and/or flexible tubing 18, resulting in a strong assembly even prior to taping. However, this abutment is removable if desired, but if present it provides added securement of wing needle assembly 12 to the skin, along with the taping of securement device 10 and the needle to the skin. For example, both axial and lateral motion of cannula 14 is suppressed by the use of this close abutment of forward strap edge 34, (only one being shown) and respective wing back edges 36.

Alignment and securement device 10 and base 23 also have a rear portion 38, also divided in this particular embodiment, which have a second, upper surface 40 that slopes downwardly to its rearward end 43, to provide a tape receiving surface 40 for securance tape that also extends over the wings of needle assembly 12. These lengths of securance tape 44, 46, are shown in FIG. 6. Each of said lengths of said securance tape 44, 46 are positioned over a different wing 20 and over a different added, upper surface 40, adhering to the patient's skin 28 at respective pairs of end sections 48 of the tape, so that there is firm securance of device 10 and the carried, winged needle set 12, using straight lengths of tape 44, 46. These are easier to apply, and require less specialized training than the conventional chevron-type tape technique and the like. Nevertheless, the pull out resistance (to the needle coming out of the skin) is generally greater than conventional needle securement techniques because the alignment and securement device 10 provides a degree of rigidity that ties together flexible tape 44, 46, flexible tube 18, flexible wings 20, and the flexible skin, so that improved safety is provided, with less effort and skill required for emplacement and securement of the needle assembly 12 on the skin. However, so immobilized is winged needle set 12 after such taping that chevron taping or other taping may be performed if desired as additional taping restraint, without fear of causing lacerating movement of the sharp distal tip of cannula 14.

Turning particularly to FIG. 5, added, upper surface 40 at the rear of strap 24 has an uppermost, maximum height portion 42 that is preferably adjacent to strap 24, and is higher than the bottom 44 of strap legs 27, where they join base 23. This provides added elevation for the tapes 44, 46 that are. extending along surface 40, to each engage with the upper surface of a wing 20 residing on first, angled, upper surface 21, without a major discontinuity being formed in the tape because of the thickness of wing 20. This added amount of elevation provided at maximum height portion 42 can thus accommodate for the thickness of the wings.

From FIG. 4, it can be seen that the bottom surface 46, under the top of U-shaped, self-supporting strap 24, can preferably abut the top surface of hub 16 and/or tube 18 to provide an added guide, security and immobilization of cannula 14 and hub 16 carried in securement device 10.

Thus, by this invention, a simple, inexpensive, easily molded securement device may be provided to support intravenous needles that must be taped onto the skin, such as hemodialysis fistula needles. The securement is easily applied with increased resistance to pull out, and less risk of internal laceration by the needle tip as the device is being taped in place. The desired angle of the needle to the skin can be reflected by first, angled, upper surface 21, to facilitate effective, inexpensive, and safe use of intravenous needles.

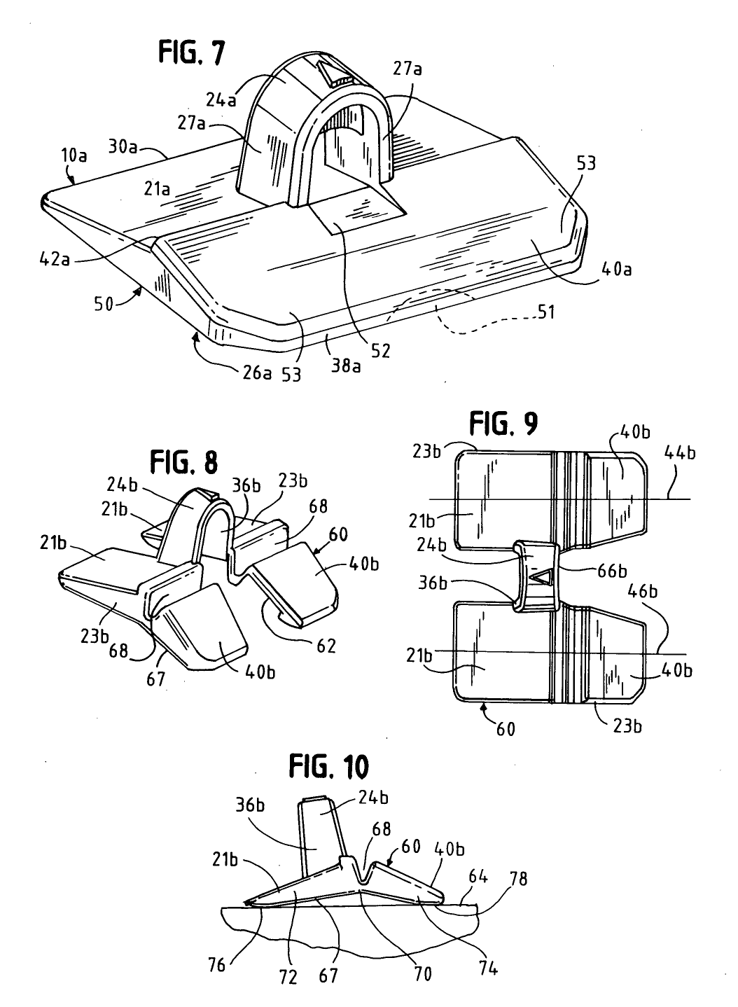

Turning to FIG. 7, another embodiment of the securement device 10a of this invention is disclosed having a U-shaped, self-supporting strap 24a as in the previous embodiment, having similar, substantially vertical legs 27a, but, in this embodiment, legs 27a join a base 50 which is unitary in a single piece, and not comprising two spaced portions as in the previous embodiment. A recess 52 may be provided in the upper surface of base 50 underneath U-shaped strap 24a, to provide desired space for a winged needle hub similar to hub 16 and/or tube similar to tube 18. Base 50, as in the previous embodiment, defines a flat bottom 26a to rest on the skin of the patient, and a unitary, first, angled, upper surface 21a which slopes downwardly to the actual forward edge 30a, which extends the entire width of securement device base 50. First, upper surface 21a slopes as does the corresponding surface 21 of the previous embodiment.

Also, an added, second, surface 40a is provided, similar to surface 40, sloping downwardly and rearwardly from U-strap 24a. The angle of slope of second, upper surface 40a may be similar to, or different from the angle of slope of first upper surface 21a. In the latter case when the angle of slope is different, in both this embodiment and the previous embodiment, the device may be reversed on the skin of the patient so that rear edges 38, 38a may be used as the front edge of the device, with the wings of a needle resting on second, upper surface 40, 40a rather than first upper surface 21, 21a, and the strips of tape adhere to the wings on surface 40, 40a while adhering to the actual surface 21, 21a itself, in a manner that is reversed from what could normally be done. By this means, if the respective upper surfaces 21, 40 or 21a, 40a have different angles to the skin, the respective surfaces will urge the wings into a desired angle which, in turn will reflect the desired needle and hub angle so that different needle angles may be provided to the needle, depending upon whether securement device 50 is placed with edge 30, 30a forward or edge 38, 38a forward.

Uppermost, maximum height portion 42a is seen, similar in structure and function of maximum height portion 42 of the previous embodiment. However, in the event that securement device 50 is intended for use facing in either direction to obtain a variation of wing and needle angles relative to the skin, the respective maximum height portions 42, 42a may be eliminated if desired, so that securement device 50 is more symmetrical, to facilitate use in either direction with either edge 30a, or 38a facing forwardly.

An optional, bottom-opening, central tunnel portion 51 may be provided, thus defining laterally spaced segments 53 in base 50, so that device 10a can be used to stabilize a blood vessel during cannulation, as described above.

The embodiment of FIG. 7 is preferably pre-engaged to the tubing similar to tubing 18 of winged needle set 12 during manufacture and may be delivered to clinician ready for sliding forward into engagement of winged needle set 12 after cannulation.

Referring to FIGS. 8-10, another embodiment of securement device 60 is disclosed. Securement device 60 defines a bifurcated base 23b, comprising two separate segments as shown, similar to base 23 of the first embodiment. As before, an inverted U-shaped, self-supporting strap 24b is provided, connecting the separate base segments 23b. Specifically, the embodiment of FIGS. 8-10 is similar to the embodiment of FIGS. 1-6, except as otherwise described herein.

Base segments 23b each define a first optionally angled upper surface 21b, and also a second optionally angled upper surface 40b, the surface angles being opposed to each other, and the same or different relative to bottom surface 67 and to the skin 64 when flat and placed thereon. As shown a central portion of bottom of surface 67 is spaced from the skin 64, while end portions 76, 78 are in contact with the skin. A plastic-saving recess 62 may optionally be placed on the underside of base segments 23b so that the flat bottom 67 is raised in central portions thereof. This may be done in the other embodiments as well.

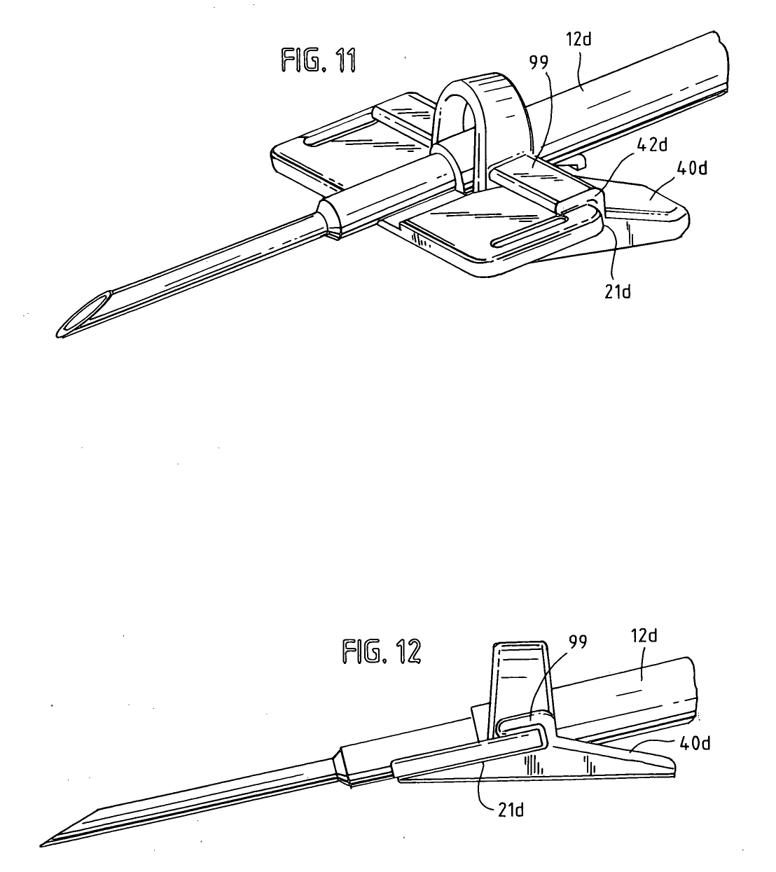

In this embodiment, upper surfaces 21 b and 40b do not need to be angled to the flat bottom surface 67 of securement device 60, although they may be so angled if desired. The reason for this is that the angle may be provided between the respective surfaces 21b, 40b and the skin 64 of a patient without regard to any angle between surfaces 21b, 40b and the bottom surface 67 of securement device 60. This is accomplished by the presence of transverse groove portion 68 formed respectively in the two segments of base 23b. Groove portion 68 thus defines a transverse line of bending weakness 70, permitting manual bending of base 23b to form two angled sections 72, 74, which are bent to an obtuse angle, or even a ninety degree angle from their original configuration, in which bottom surface 67 may be flat, although it also may be molded in bent manner if desired. Thus, the angle formed between the bottom surfaces 67 of the respective angled sections 72, 74 may be adjusted by bending, so that the angle of first upper surface(s) 21b, and also second upper surface(s) 40b may be respectively adjusted, relative to the skin 64 of a patient on which device 60 rests, as shown in FIG. 10.

Accordingly, the angle of either surface 21b or 40b may thus be adjusted by bending at the angle formed at transverse line of bending weakness 70 so that a wide range of angles of surfaces 41b, 40b to skin 64 may be achieved. Then, securement device 60 may be positioned and taped in position in a manner similar to that previously described, being retained there in the desired angle by the tape. A winged needle may rest on securement device 60 as in the previous embodiment, with the respective wings resting on the two surface portions 21b, or resting on surface portions 40b if desired, to support the needle as it penetrates the skin at a desired, predetermined angle. Taping of the wings to device 60, and taping the device and wings to the skin, as in the previous embodiment, secures the entire system so that the cannula is relatively rigidly held in position so that internal laceration of a blood vessel (or graft) in which the sharp cannula tip resides is less likely to take place.

As before, space 66b between the respective portions of base 23b is preferably proportioned to snugly fit a needle hub or tubing adjacent to the needle hub, to restrain motion of the needle hub and needle in vertical and horizontal movement. The abutment of legs 36b of strap 24b, engaging the rear end of the needle wings adjacent to the hub, restricts longitudinal motion. Then, the entire system is taped in place, preferably with longitudinal lengths of tape 44b, 46b, schematically illustrated as lines, to secure the needle wings to surfaces 21b, and to secure the tape to surfaces 40b, to tape the entire system to the skin, as in previous embodiments, but with portions of base 23b defining typically an obtuse angle to each other, as shown in FIG. 10, to provide the best angle of outer surfaces 21b or 40b for retaining needle wings in a desired position.

FIGS. 11 and 12 show another embodiment that is similar to the FIG. 5 embodiment, except as noted. Connected to maximum height portion 42d and extending toward the forward edges of first, angled upper portions 21d are forward cover portions 99. In use, the wings of winged needle set 12d are inserted between first, angled upper portions 21d and forward cover portions 99. Thus, the winged needle set 12d is secured without the need for tape directly touching the wing. Rather, tape is preferably secured only to the skin and the top of forward cover portions 99 and typically second, upper surfaces 40d.

Thus, a needle securement device, or simply a needle alignment device for protection to the nurse who inserts the needle, may be provided by this invention, to significantly facilitate the insertion of a needle through the skin and the maintenance of the needle in position while reducing the risk of injury.

The above has been offered for illustrative purposes only, and is not intended to limit the scope of the invention of this application, which is as defined in the claims below.

1. A securement device for an intravascular, winged needle set, which comprises:
an inverted U-shaped, self-supporting strap, said strap having legs attached to base, said base having a bottom to rest on the skin of a patient, a portion of said base being forward of said strap and having a first, upper surface that slopes downwardly to a forward end at an angle of essentially 5-30°.

2. The securement device of claim 1 in which the upper surface slopes downwardly at an angle of essentially 5-10°.

3. The securement device of claim 1 in which said base is divided into a pair of spaced segments with a space extending under said U-shaped strap, said space being of a width to at least partly receive a hub of said winged, intravascular needle.

4. The securement device of claim 1 which has a winged needle hub positioned under said strap with wings of said needle set resting on said first, upper surface.

5. The securement device of claim 4 in which forward edge portions of the strap are in abutting relation with said wings next to the hub.

6. The securement device of claim 1 in which a portion of said base behind said strap has a second, upper surface, to provide a tape receiving surface for securance tape that also extends over the wings of a needle set carried on said securement device.

7. The securement device of claim 6 in which said second, upper surface has a maximum height portion that is adjacent to said strap, and is higher than the bottom of said strap legs.

8. The securement device of claim 6, secured to the skin of a patient with separate lengths of securance tape each extending over the tape receiving surface and a wing of said winged needle set and said securance tape being sufficiently long to secure the needle set and at least one of said wings to the skin of the patient.

9. The securement device of claim 8 in which said lengths of tape extend in a direction generally longitudinal to the axis of said needle set, said lengths of tape having opposed end sections adhering to the skin.

10. The securement device of claim 8 in which said lengths of tape are attached to upper surfaces of a pair of wings, a and spaced, tape receiving surface portions, and the skin.

11. The securement device of claim 1, in which separate lengths of securance tape each extend over a wing of a winged needle set carried on said securement device, said lengths of tape extending in a direction generally longitudinal to the axis of said needle set on either side of the inverted, U-shaped strap, said lengths of tape having opposed end sections adhering to the skin.

12. A securement device for intravascular, winged needle set, which comprises:
an inverted U-shaped, self-supporting strap, said strap having legs attached to a a base, said base having a bottom to rest on the skin of a patient, a portion of said base forward of said strap having a first, upper surface that slopes downwardly to a forward end at an angle of essentially 5-30°, said base being divided into a pair of spaced segments with the space between said segments extending under said U-shaped strap, said space being of a width to receive a hub of said winged, intravascular needle set.

13. The securement device of claim 12 in which a portion of said base behind said strap has a second, upper surface that slopes downwardly to a rearward end, to provide a tape receiving surface for securance tape that also extends over the wings of a needle set carried on said securement device.

14. The securement device of claim 13 in which said second upper surface has a maximum height portion that is adjacent to said strap, and is higher than the bottom of said strap legs.

15. The securement device of claim 12 which has a winged needle hub positioned under said strap with wings of said needle set resting on said first, upper surface and the needle set having a cannula penetrating the skin of patient.

16. The securement device of claim 12, secured to the skin of a patient with separate lengths of securance tape each extending over and adhering to the second, upper surface and a wing of said winged needle set, while extending in a direction generally longitudinal to the axis of said needle set, said lengths of tape each having opposed end sections adhering to the skin.

17. The securement device of claim 13 in which the second, upper surface slopes at a different angle from the first upper surface to permit reversal of functions of the first and second upper surfaces.

18. The securement device of claim 6 in which the second, upper surface slopes at a different angle from the first upper surface, to permit reversal of functions of the first and second upper surfaces.

19. The securement device of claim 18 in which the second, upper surffice slopes at an angle of essentially 10° to 15°, and the first, upper surface slopes at a lesser angle.

20. The securement device of claim 12 in which said base defines a transverse line of bending weakness to permit manual bending of the base to form two angled sections from said flat bottom, to adjust the angle of said first, upper surface to the skin of a patient on which said device rests.

21. A stabilization device for cannulation of a vein, fistula, graft or the like, which comprises:
an inverted U-shaped, self-supporting strap, said strap having legs attached to a base, said base having a bottom and a first, upper surface, and being divided into a pair of spaced segments with a space extending under said U-shaped strap, said space being of a width capable of straddling at least a portion of a subcutaneous artery, vein, fistula, graft or like vessel when the spaced portions are pressed down on the skin adjacent to such vessel.

22. The securement device of claim 21 in which said base defines a transverse line of bending weakness to permit manual bending of the base to form two angled sections from said bottom, to adjust the angle of said first, upper surface to the skin of a patient on which said device rests.

23. A securement device for an intravascular, winged needle set, which comprises:
an inverted U-shaped, self-supporting strap, said strap having substantially vertical legs attached to a center portion of a base, said base having a bottom to rest on the skin of a patient, said base further defining a transverse line of bending weakness to permit manual bending of the base to form two angled sections from said bottom to adjust the angle of an upper surface of said base to the skin of a patient on which said device rests.

24. The securement device of claim 23 in which said base is also divided into a pair of spaced segments with the space extending under said U-shaped strap, said space being of a width to at least partly receive a hub of said winged, intravascular needle set.

25. The securement device of claim 23 in which said transverse line of bending weakness is manually bent while said device rests on the skin of a patient, so that a central portion of said base does not rest on the skin of the patient, while end portions of said base do rest on the skin of a patient, said device being taped to the skin of the patient.

26. A device for securing by strips of adhesive tape a winged needle set, that penetrates the skin of a patient against motion of the cannula of said set, within the skin, which comprises:
a base for placement on the skin of the patient, and for immovably holding the winged needle, while said cannula penetrates the skin and at least a portion of the wings of the needle are separated from the skin by the device.

27. The device of claim 26, placed on the skin of a patient and carrying said winged needle set with the needle penetrating the skin, said base having upper surfaces that support the needle wings.

28. The device of claim 27 in which said device and said winged needle set are secured to the skin by strips of adhesive tape overlaying the device and/or wings.

29. The method of stabilizing a blood vessel to facilitate its penetration by a cannula through the skin, which comprises:
applying to the skin a stabilization device which comprises a base with the blood vessel positioned under the center of the base to limit lateral movement of said blood vessel as a needle is advanced to penetrate the blood vessel through the skin.

30. The method of claim 29 wherein the base comprises a pair of spaced base segments, with the blood vessel positioned between said base segments.

31. The method of claim 30 in which said stabilization device is thereafter positioned to support and stabilize the winged needle set as it resides on the skin in skin-penetrating relation, further including the step of taping a winged needle set and the stabilization device to the skin.

32. The method of claim 29 in which said stabilization device is thereafter positioned to support and stabilize the winged needle set as it resides on the skin in skin-penetrating relation, further including the step of taping a winged needle set and the stabilization device to the skin.

33. The method of claim 31 in which wings of the winged needle set rest, after said taping, on said base segments, at least a portion of said wings being spaced from the skin.

34. The method of stabilizing a cannula residing in a blood vessel and extending through the skin of a patient, said cannula having a hub, which comprises:
placing a securement device comprising a base onto the skin of the patient adjacent to said cannula hub; and taping the skin, the base, and the cannula hub together into a substantially rigid composite.

35. The method of claim 33 in which said hub has wings that at least mostly rest on said base, spaced from the skin.

36. The securement device of claim 1 in which the legs of said strap are substantially vertical and attached to a center portion of said base.

37. The securement device of claim 12 in which the legs of said strap are substantially vertical and attached to a center portion of said base.

38. The securement device of claim 6 in which the second, upward surface slopes downwardly to a rearward end.
